(19) United States
(12) Patent Application Publication
(10) Pub. No.: US 2015/0359591 A1
(43) Pub. Date: Dec. 17, 2015

(54) SYSTEM AND METHOD FOR CONTROLLING ENERGY-BASED TREATMENT HANDPIECES

(71) Applicant: LUMENIS LTD., Yokneam Ilit (IL)

(72) Inventors: Eyal Benisty, Kfar Hachoresh (IL); Issar Yazbin, Rehovot (IL); Shlomi B. Naim, Migdal Haemek (IL)

(21) Appl. No.: 14/718,526

(22) Filed: May 21, 2015

Related U.S. Application Data
(60) Provisional application No. 62/003,954, filed on May 28, 2014.

(51) Int. Cl. A61B 18/20

(52) U.S. Cl. CPC A61B 18/203; A61B 2018/2085; A61B 2018/00875

(57) ABSTRACT

A method for controlling the firing of an energy device when placed in contact with skin tissue includes providing an energy apparatus having a distal portion and configured to be fired; a controller configured to initiate the firing of the energy apparatus; providing a tip device having a proximal portion in contact with the distal portion of the energy apparatus and a distal portion for contacting the skin tissue; and, providing at least one sensor, the at least one sensor being normally unactivated but being activated when the tip device comes into contact with the skin tissue; and, activating the sensor by pressing the tip device onto the skin tissue, the activation causing the controller to fire the energy apparatus.

### RELATED APPLICATIONS

The present application is related to and claims priority to U.S. Provisional Application Ser. No. 62/003,954, filed May 28, 2014, the total disclosure of which is herein incorporated by reference.

### FIELD OF THE INVENTION

The present invention relates to an apparatus and a method for controlling the activation of an energy source in a treatment handpiece in general, including any type of medical or aesthetic device which comes into contact with human tissue and more particularly the firing of laser devices, including a laser scanner.

### BACKGROUND OF THE INVENTION

Laser energy is often used for aesthetic applications such as hair removal, scar and acne treatment or skin tightening.

For convenience of operation, laser devices may be supplied with an arm, umbilical cord or a fiber connected to a handpiece. The handpiece may comprise a tip, either disposable or reusable, which confines or directs the laser beam to a selected area, such as the area to be treated. One such tip is shown in U.S. Design Pat. No. 621,306, which is assigned to the assignee of the present invention. The entirety of such design patent is incorporated by reference herein.

In use, the user holds the handpiece, positions it on or directs it to the desired treatment area and presses an on/off button in the handpiece or a footswitch (hereinafter generally may be referred to as a “trigger”) to apply the laser energy, the on/off button being shown in FIG. 4 as element 105 and the footswitch as element 107. Once application of the laser beam energy is completed, the user turns the system off by releasing the button in the handpiece or by releasing the footswitch trigger, then moves the handpiece to another position or in another direction, and repeats the process. This necessitates the user to press and depress a trigger, an on/off button or a footswitch many times in synchronization with the handpiece movement and positioning on or towards the patient's skin. In addition, it has been found that the desired clinical effect can be best achieved if the user holds the handpiece steady and in a position generally orthogonal to the skin tissue to be treated while firing the laser. However, no feedback mechanism is currently available for assuring the steady and orthogonal positioning of the handpiece.

### SUMMARY OF THE PRESENT INVENTION

In an aspect, a device for controlling the firing of an energy device when placed in contact with skin tissue includes an energy apparatus having a distal portion and configured to be fired; a controller configured to initiate the firing of the energy apparatus; a tip device having a proximal portion in contact with the distal portion of the energy apparatus and a distal portion for contacting the skin tissue; and, at least one sensor, the at least one sensor being normally unactivated but being activated when the tip device comes into contact with the skin tissue.

In a second aspect, the at least one sensor is positioned between the distal portion of the energy apparatus and the proximal portion of the tip device.

In a third aspect, the energy apparatus is one or more of: a laser device; an intense pulsed light device; a RF device and an ultrasound device. The at least one sensor includes two or more sensors, wherein the two or more sensors are activated when the position of the energy apparatus is approximately orthogonal to the skin tissue.

In another aspect, the tip device is one or removable or permanently affixed to the distal portion of the energy apparatus. The tip device is one or reusable or disposable.

In yet another aspect, the device includes a spring between the distal portion of the energy apparatus and the proximal portion of the tip device, the spring biasing the tip device away from the energy apparatus such that the sensor is unactivated until the tip device is pressed against the skin tissue and overcomes the biasing of the spring, activating the sensor. Upon activation of the sensor, the controller causes the firing of the energy apparatus.

In yet a further aspect, the device includes one of a foot switch or a switch on the energy apparatus, and wherein the controller causes the firing of the energy apparatus only when the sensor is activated and when one of the foot switch or the switch on the energy apparatus is pressed.

In another aspect, a method for controlling the firing of an energy device when placed in contact with skin tissue includes: providing an energy apparatus having a distal portion and configured to be fired; a controller configured to initiate the firing of the energy apparatus; providing a tip device having a proximal portion in contact with the distal portion of the energy apparatus and a distal portion for contacting the skin tissue; and, providing at least one sensor, the at least one sensor being normally unactivated but being activated when the tip device comes into contact with the skin tissue; and, activating the sensor by pressing the tip device onto the skin tissue, the activation causing the controller to fire the energy apparatus. The energy apparatus is one or more of: a laser device; an intense pulsed light device; a RF device or an ultrasound device.

### BRIEF DESCRIPTION OF THE DRAWINGS

FIG. 1. is a schematic drawing of handpiece, laser and tip, with a switch sensor for activating laser scanner.

FIG. 2. is a schematic drawing of handpiece, laser and tip, with multiple switch sensors for activating the laser.

FIG. 3. shows a side view of handpiece, tip, switch sensor and spring.

FIG. 4 illustrates a system including the handpiece, a footswitch, and a controller.

### DETAILED DESCRIPTION OF THE INVENTION

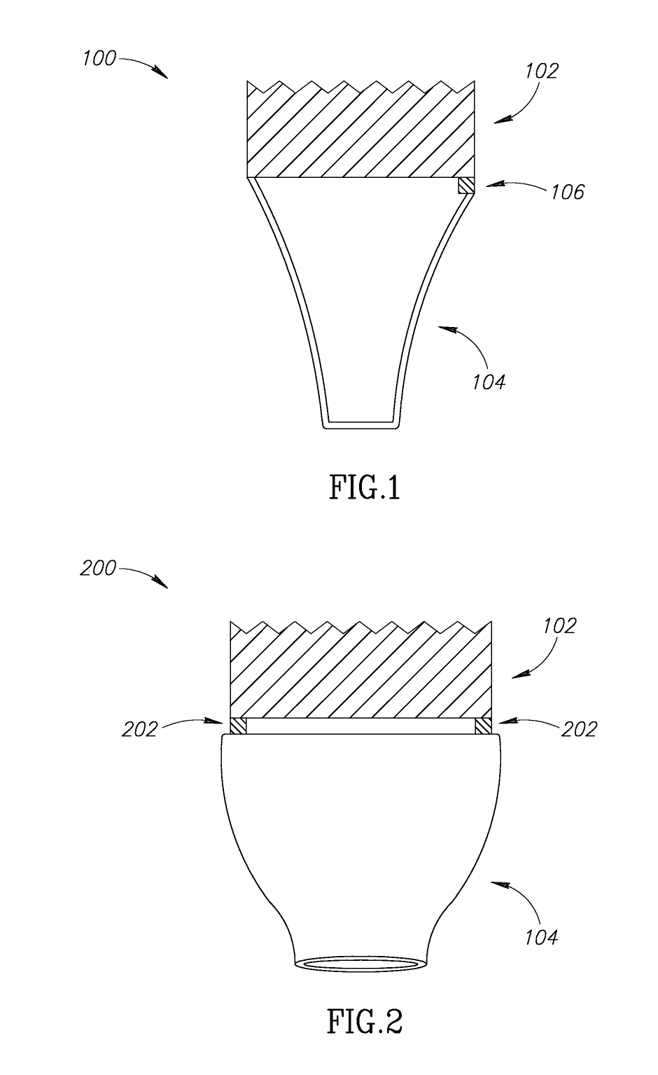

FIG. 1 shows one embodiment for a handpiece 100. The handpiece 100 is composed of a laser device 102 connected to a tip 104. The laser device 102 may be set to fire the laser in a defined pattern. The tip 104 can be either disposable or reusable. At least one mechanical switch or proximity sensor or other sensor 106 can be positioned between the distal end of laser 102 and the tip 104 in any of the two embodiments described here: The at least one switch sensor or other sensor 106 may be an integral part of the laser 102 and may have an interface to accept the tip 104, or, the at least one switch sensor 106 is part of the tip 104 and is connected to through suitable connections to a controller 111 shown in FIG. 4 contained in the main laser apparatus and to the laser device (which may be a laser scanner) 102 through suitable electrical connections to cause the switch to close as will be described below.

The mechanism of action of the handpiece 100 will now be further described. The user holds the handpiece 100 and presses the distal end of the tip 104 against the patient's skin.

When the tip 104 comes into contact with skin tissue within specified pressure limits, the at least one switch sensor 106 may activate the laser 102 in one of two different mode of actions: (a) the at least one switch sensor 106 may replace any other on/off switch, such as the footswitch described above, and initiate laser firing through laser 102 on the treated area by its own action; or, (b). if a safety mechanism is desired, in addition to the closing of the switch sensor, a footswitch or an on/off integrated button in the handpiece, may also be required to be pressed to cause the firing of the laser when the tip 104 is pressed and the at least one switch sensor 106 is activated.

In addition, the at least one switch sensor 106 may also include an accelerometer to sense and provide feedback of the amount of force applied when the tip is pressed against the patient's skin tissue . When the force applied exceeds a set and defined predetermined threshold, the system may be activated. Thus, the laser 102 will not fire laser energy if the handpiece 100 is not steadily positioned and not pressed with the predetermined force on the patient's skin tissue. According to one embodiment of the invention, sensor 106 may have a single threshold above which the energy source is activated. According to another embodiment of the invention, sensor 106 may have a dynamic range in which it feedbacks to the main controlling system of the energy source in proportion to the amount of pressure applied by the user. According to this embodiment, a user may select, through a user interface, which parameter characterizing the energy source or the treatment such as, but not limited to, energy level, pulse repetition rate, speed of scanning, density of a fractional treatment pattern, maybe controlled by the sensor's 106 dynamic feedback to dynamically apply such a parameter within a range of operation. By way of example, the trigger or footswitch may be the footswitch disclosed in pending U.S. patent application Ser. No. 13/038,773, the contents of which are herein incorporated by reference. For example, a user may choose to dynamically control the energy level. Once chosen, per this non limiting example, during the application of the handpiece on the tissue the handpiece will apply low energy level when the pressure is low but above a certain predefined threshold and the applied energy level may increase as the handpiece is pushed with greater force against the skin tissue.

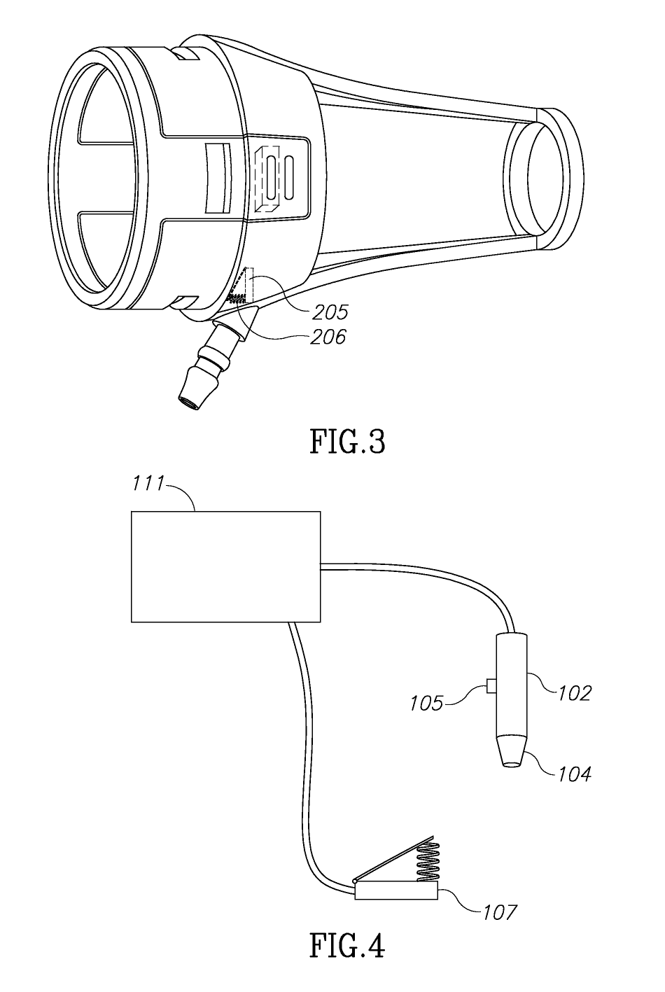

FIG. 2 shows another embodiment of a handpiece 200 with multiple switch sensors 202. The multiple switch sensors 202 are either part of the laser device or scanner 102 or the tip 104. The user may press the tip 104 against the patient's skin, and o the multiple switch sensors 202 may sense the force applied by the user to the skin tissue. The multiple switch sensors 202 may be set to activate the laser when the forces sensed by each sensor are equal and above a defined threshold. This mechanism controls the user's ability to fire laser beams only when the handpiece is orthogonally pressed against the skin surface. According to another embodiment of the present invention, one or more flexible elements, such as but not limited to a spring, may be positioned between tip 104 and handpiece 200. Spring 206 (such as that shown in FIG. 3, discussed below) may enhance the degree of freedom between tip 104 and handpiece 200 and may also enhance the user experience for sensing the dynamic range of sensor 106.

Another variation to control the firing of the laser is to incorporate a known mercury switch into the handpiece. Such mercury switches work on the principle that two end wires are not electrically connected but become electrically connected when mercury, a conductive metal in liquid form at room temperatures, flows due to positioning of the handpiece to cover both wire ends, thus closing an electrical circuit.

Another variation is to incorporate a reed switch, which reacts with a magnet, and can be activated as a result of proximity to such magnet.

Turning now to FIG. 3, this figure shows an implementation of the device of the present invention on the disposable tip shown in the aforesaid US Design Pat. No. D621,306. This embodiment includes not only a reed switch 205 as just described (or a sensor such as sensor 106 as previously described in reference to FIG. 1 or a mercury switch as described above) but also a spring 206 which operates normally in a state to push the tip in a distal direction such that the pressure of the tip against the skin tissue will overcome the resistance of the spring and cause the reed switch 205 or the sensor 106 or the mercury switch to be activated and thus cause the laser to fire. A hinge (not shown) may be included at a position opposite the reed switch/sensor and spring to allow for movement of the tip against the spring.

With the embodiments of FIGS. 1 to 3, the respective sensor or sensors or reed switch may be located either on the laser body 102 or the tips 104 themselves.

It is also understood that while a light-based laser device has been described, any other type of energy delivery device may be utilized with the present invention, including a RF device, an ultrasound device or an intense pulsed light device or any combination thereof

What is claimed is:

1. A device for controlling the firing of an energy device when placed in contact with skin tissue comprising:
an energy apparatus having a distal portion and configured to be fired;
a controller configured to initiate the firing of the energy apparatus;
a tip device having a proximal portion in contact with the distal portion of the energy apparatus and a distal portion for contacting the skin tissue; and,
at least one sensor, the at least one sensor being normally unactivated but being activated when the tip device comes into contact with the skin tissue with a predetermined amount of force.

2. The device of claim wherein the at least one sensor is positioned between the distal portion of the energy apparatus and the proximal portion of the tip device.

3. The device of claim 1 wherein the energy apparatus is one or more of: a laser device; an intense pulsed light device; a RF device and an ultrasound device.

4. The device of claim 1 wherein the at least one sensor comprises two or more sensors, wherein the two or more sensors are activated when the position of the energy apparatus is approximately orthogonal to the skin tissue.

5. The device of claim 1 wherein the tip device is one or removable or permanently affixed to the distal portion of the energy apparatus.

6. The device of claim 1 wherein the tip device is one or reusable or disposable.

7. The device of claim 1 further comprising a spring between the distal portion of the energy apparatus and the proximal portion of the tip device, the spring biasing the tip device away from the energy apparatus such that the sensor is unactivated until the tip device is pressed against the skin tissue with a predetermined amount of force and overcomes the biasing of the spring, activating the sensor.

8. The device of claim 1, wherein upon activation of the sensor, the controller causes the firing of the energy apparatus.

9. The device of claim 8, further comprising one of a foot switch or a switch on the energy apparatus, and wherein the controller causes the firing of the energy apparatus only when the sensor is activated and when one of the foot switch or the switch on the energy apparatus is pressed.

10. A method for controlling the firing of an energy device when placed in contact with skin tissue comprising:
providing an energy apparatus having a distal portion and configured to be fired;
a controller configured to initiate the firing of the energy apparatus;
providing a tip device having a proximal portion in contact with the distal portion of the energy apparatus and a distal portion for contacting the skin tissue; and,
providing at least one sensor, the at least one sensor being normally unactivated but being activated when the tip device comes into contact with the skin tissue; and,
activating the sensor by pressing the tip device onto the skin tissue, the activation causing the controller to fire the energy apparatus.

11. The apparatus of claim 10, wherein the energy apparatus is one or more of: a laser device; an intense pulsed light device; a RF device or an ultrasound device.

12. The apparatus of claim 1, wherein the sensor is one of a proximity sensor, a reed switch or a mercury switch.
